(19) United States
(12) Patent Application Publication
(10) Pub. No.: US 2021/0108187 A1
(43) Pub. Date: Apr. 15, 2021

(54) BOTULINUM NEUROTOXINS PRODUCTION METHODS

(71) Applicant: Bonti, Inc., Madison, NJ (US)

(72) Inventors: Satish Menon, Madison, NJ (US); Michael Jarpe, Madison, NJ (US); Kenton Able, Madison, NJ (US)

(21) Appl. No.: 16/608,360

(22) PCT Filed: Apr. 27, 2018
(86) PCT No.: PCT/US2018/029855

§ 371 Date: Oct. 25, 2019

Related U.S. Application Data
(60) Provisional application No. 62/525,073, filed on Jun. 26, 2017.
(60) Provisional application No. 62/525,062, filed on Jun. 26, 2017.
(60) Provisional application No. 62/525,050, filed on Jun. 26, 2017.
(60) Provisional application No. 62/491,376, filed on Apr. 28, 2017.

(51) Int. Cl. C12N 9/64

(52) U.S. Cl. CPC C12N 9/6489; C12Y 304/24069

(57) ABSTRACT

Disclosed herein are systems and methods for manufacturing botulinum neurotoxin serotype E (BoNT/E) with improved yield and purity of BoNT/E. Disclosed herein are systems and methods for manufacturing BoNT/E drug substance.

### FIELD

The present specification relates to the production of botulinum neurotoxins and cells, including banking of cells.

### BACKGROUND

Botulinum toxins are the most potent protein toxins. They act by blocking acetylcholine release at the neuromuscular junction, resulting in denervation of muscles. Botulinum toxins also have activity at other peripheral cholinergic nerve terminals and lead, for example, to reduced salivation or sweating and to diminished facial lines and wrinkles. Due to their specificity of action, the range of clinical applications of botulinum toxins is continuously growing, and botulinum toxins are today being used extensively as pharmaco-cosmetics.

The botulinum toxins are synthesized and released by certain Clostridium species in the form of large complexes comprising the botulinum toxin molecule (the “neurotoxic component”) and associated non-toxic bacterial proteins (also referred to as “complexing proteins”). The complexing proteins include different non-toxic hemagglutinin (HA) proteins and non-toxic non-hemagglutinin (NTNH) proteins. The molecular weight of the toxin complex varies among the seven distinct botulinum toxin serotypes (A, B, C, D, E, F and G) from about 300 kDa to about 900 kDa. The complexing proteins provide stability to the neurotoxic component.

Regardless of serotype, the molecular mechanism of toxin intoxication appears to be similar and to involve at least three steps or stages. In the first step of the process, the toxin binds to the presynaptic membrane of the target neuron through a specific interaction between the heavy chain, H chain, and a cell surface receptor; the receptor is thought to be different for each type of botulinum toxin and for tetanus toxin. The carboxyl end segment of the H chain, H_{C}, appears to be important for targeting of the toxin to the cell surface.

In the second step, the toxin crosses the plasma membrane of the poisoned cell. The toxin is first engulfed by the cell through receptor-mediated endocytosis, and an endosome containing the toxin is formed. The toxin then escapes the endosome into the cytoplasm of the cell. This step is thought to be mediated by the amino end segment of the H chain, H_{N}, which triggers a conformational change of the toxin in response to a pH of about 5.5 or lower. Endosomes are known to possess a proton pump which decreases intra-endosomal pH. The conformational shift exposes hydrophobic residues in the toxin, which permits the toxin to embed itself in the endosomal membrane. The catalytic domain (light chain) then translocates through the endosomal membrane into the cytoplasm.

Type E botulinum neurotoxin (BoNT/E) requires activation during manufacture. In the wild, BoNT/E is activated with trypsin in the digestive system after the animal has ingested contaminated food. Trypsin is a serine protease from the PA clan superfamily, found in the digestive system of many vertebrates, where it hydrolyses proteins. Trypsin is formed in the small intestine when its proenzyme form, the trypsinogen produced by the pancreas, is activated. Trypsin cleaves peptide chains mainly at the carboxyl side of the amino acids lysine or arginine, except when either is followed by proline. It is used for numerous biotechnological processes. The process is commonly referred to as trypsin proteolysis or trypsinisation, and proteins that have been digested/treated with trypsin are said to have been trypsinized.

### SUMMARY

Disclosed herein are compositions and methods for use in producing clostridial cells. In embodiments, the clostridial cells comprise botulinum cells.

Disclosed herein are compositions and methods for use in banking clostridial cells. In embodiments, the clostridial cells comprise botulinum cells.

Disclosed herein are compositions and methods for use in producing and purifying clostridial neurotoxins. In embodiments, the clostridial neurotoxins comprise botulinum toxins, for example “fast-acting” botulinum toxins such as BoNT/E.

Disclosed herein are compositions and methods for use in producing and purifying clostridial neurotoxins. In embodiments, the clostridial neurotoxins comprise botulinum toxins, for example “fast-recovery” botulinum toxins such as BoNT/E.

Disclosed herein are systems and methods for manufacturing BoNT/E with improved toxin production during the fermentation step in the manufacturing process.

Disclosed herein are systems and methods for manufacturing BoNT/E with a minimal residual trypsin level.

In embodiments, disclosed treatment methods comprise administration of a fast-acting botulinum neurotoxin to a patient.

In embodiments, the patient is neurotoxin naïve.

In embodiments, the patient is clostridial toxin naïve.

In embodiments, the patient is botulinum toxin naïve.

In embodiments, the patient is BoNT/E naïve.

In embodiments, the patient is botulinum type A (BoNT/A) naïve.

In embodiments, the patient is botulinum type B (BoNT/B) naïve.

In embodiments, the patient is “fast-acting” neurotoxin naïve.

In embodiments, the patient is “fast-recovery” neurotoxin naïve.

Disclosed embodiments comprise wild-type neurotoxins, for example wild-type clostridial neurotoxins, for example wild type botulinum toxins, such as BoNT/E.

In embodiments, neurotoxin dosage is expressed in protein amount.

Embodiments comprise methods for creating a Clostridium botulinum master cell bank and improving yields and purity of BoNT/E during fermentation and purification.

### BRIEF DESCRIPTION OF THE DRAWINGS

FIG. 1 depicts injection sites used in a cosmetic surgery procedure.

FIG. 2 shows primary efficacy of a glabellar line treatment study.

FIG. 3 shows secondary efficacy of a glabellar line treatment study.

FIG. 4 shows the effect of a single local administration of a disclosed BoNT/E composition in a Brennan rat model of post-operative pain.

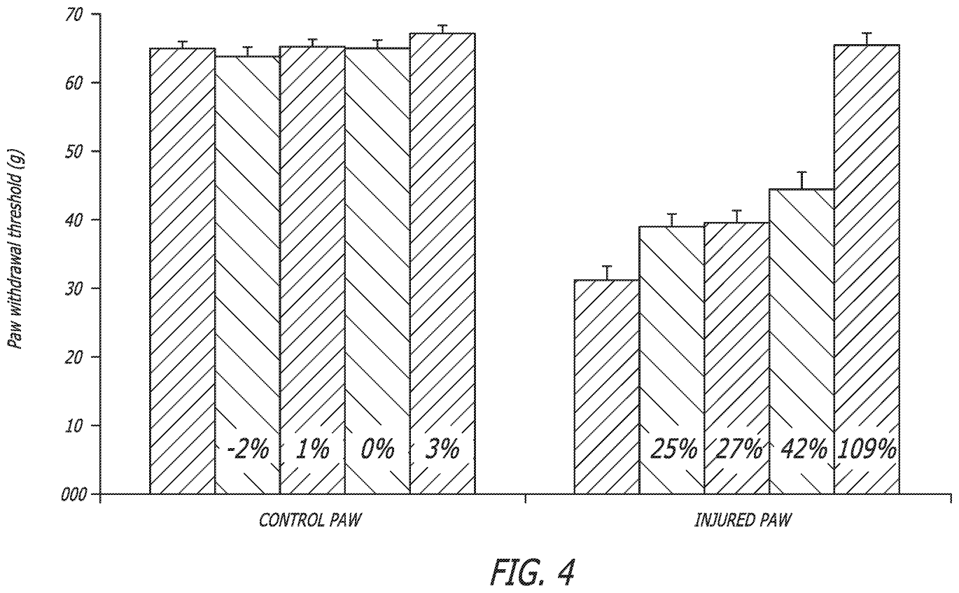

### DETAILED DESCRIPTION

Disclosed herein are compositions, systems, and methods for use in producing clostridial cells. In embodiments, the clostridial cells comprise botulinum cells, for example botulinum cells that produce BoNT/E.

Disclosed herein are compositions, systems, and methods for use in banking clostridial cells. In embodiments, the clostridial cells comprise botulinum cells, for example botulinum cells that produce BoNT/E.

Disclosed herein are compositions, systems, and methods for use in producing and purifying clostridial neurotoxins. In embodiments, the clostridial neurotoxins comprise botulinum toxins, for example “fast-acting” botulinum toxins such as BoNT/E.

Disclosed herein are compositions, systems, and methods for use in producing and purifying clostridial neurotoxins. In embodiments, the clostridial neurotoxins comprise botulinum toxins, for example “fast-recovery” botulinum toxins such as BoNT/E.

Disclosed herein are systems and methods for manufacturing BoNT/E with improved toxin production during the fermentation step in the manufacturing process.

Disclosed herein are systems and methods for manufacturing BoNT/E with a minimal residual trypsin level.

Embodiments disclosed herein can comprise administration of clostridial neurotoxins, for example botulinum neurotoxins such as BoNT/E. Such administration can reduce local autonomic nerve activity.

In embodiments, compositions disclosed herein can comprise a fast-acting botulinum toxin, for example, BoNT/E.

In embodiments, compositions disclosed herein can comprise a fast-recovery botulinum toxin, for example, BoNT/E.

In embodiments, compositions disclosed herein can comprise fast acting, fast-recovery botulinum toxins, for example, BoNT/E.

Disclosed embodiments comprise wild-type neurotoxins, for example wild-type BoNT/E.

In embodiments, methods disclosed herein can comprise dosages sufficient to inhibit nerve activity.

In embodiments, methods disclosed herein can comprise dosages sufficient to inhibit muscle contraction.

In embodiments, methods disclosed herein can comprise dosages insufficient to inhibit muscle contraction.

In embodiments, neurotoxin dosage is expressed in protein amount, for example nanograms (ng).

Embodiments comprise use of disclosed compositions and methods in conjunction with a surgical procedure, for example a cosmetic procedure.

### Definitions

“Administration,” or “to administer” means the step of giving (i.e. administering) a pharmaceutical composition or active ingredient to a subject. The pharmaceutical compositions disclosed herein can be administered via a number of appropriate routs, however as described in the disclosed methods, the compositions are locally administered by e.g. intramuscular routes of administration, such as by injection or use of an implant.

“Botulinum toxin” or “botulinum neurotoxin” means a neurotoxin derived from Clostridium botulinum, as well as modified, recombinant, hybrid and chimeric botulinum toxins. A recombinant botulinum toxin can have the light chain and/or the heavy chain thereof made recombinantly by a non-clostridial species. “Botulinum toxin,” as used herein, encompasses the botulinum toxin serotypes A, B, C, D, E, F, G and H. “Botulinum toxin,” as used herein, also encompasses both a botulinum toxin complex (i.e. the 300, 600 and 900 kDa complexes) as well as pure botulinum toxin (i.e. the about 150 kDa neurotoxic molecule), all of which are useful in the practice of the present invention. “Purified botulinum toxin” means a pure botulinum toxin or a botulinum toxin complex that is isolated, or substantially isolated, from other proteins and impurities which can accompany the botulinum toxin as it is obtained from a culture or fermentation process. Thus, a purified botulinum toxin can have at least 95%, and more preferably at least 99% of the non-botulinum toxin proteins and impurities removed.

“Biocompatible” means that there is an insignificant inflammatory response at the site of implantation of an implant.

“Clostridial neurotoxin” means a neurotoxin produced from, or native to, a Clostridial bacterium, such as Clostridium botulinum, Clostridium butyricum or Clostridium beratti, as well as a Clostridial neurotoxin made recombinantly by a non-Clostridial species.

“Drug product” or “DP” has the meaning as set forth in the Code of Federal Regulations. 21CFR314.3, as revised Apr. 1, 2015, states that drug product means a finished dosage form, for example, tablet, capsule, or solution, that contains a drug substance, generally, but not necessarily, in association with one or more other ingredients.

“Drug substance” or “DS” has the meaning as set forth in the Code of Federal Regulations. 21CFR314.3, as revised Apr. 1, 2015, states that drug substance means an active ingredient that is intended to furnish pharmacological activity or other direct effect in the diagnosis, cure, mitigation, treatment, or prevention of disease or to affect the structure or any function of the human body, but does not include intermediates used in the synthesis of such ingredient.

“Entirely free” (“consisting of” terminology) means that within the detection range of the instrument or process being used, the substance cannot be detected or its presence cannot be confirmed.

“Essentially free” means that within the detection range of the instrument or process being used, only trace amounts of the substance can be detected.

“Fast-acting” as used herein refers to a botulinum toxin that produces effects in the patient more rapidly than those produced by, for example, a BoNT/A. For example, the effects of a fast-acting botulinum toxin can be visible within 36 hours, 40 hours, 44 hours, 48 hours, 52 hours, 56 hours, 60 hours, or the like.

“Fast-recovery” as used herein refers to a botulinum toxin that whose effects diminish in the patient more rapidly than those produced by, for example, a BoNT/A. For example, the effects of a fast-recovery botulinum toxin can diminish within, for example, 120 hours, 150 hours, 300 hours, 350 hours, 400 hours, 500 hours, 600 hours, 700 hours, 800 hours, or the like. It is known that botulinum toxin type A can have an efficacy for up to 12 months. However, the usual duration of an intramuscular injection of a botulinum neurotoxin type A is typically about 3 to 4 months.

“Intermediate-acting” as used herein refers to a botulinum toxin that produces effects more slowly that a fast-acting toxin.

“Neurotoxin” means a biologically active molecule with a specific affinity for a neuronal cell surface receptor. “Neurotoxin” includes Clostridial toxins both as pure toxin and as complexed with one to more non-toxin, toxin associated proteins.

“Patient” means a human or non-human subject receiving medical or veterinary care.

“Pharmaceutical composition” means a formulation in which an active ingredient can be a botulinum toxin. The word “formulation” means that there is at least one additional ingredient (such as, for example and not limited to, an albumin [such as a human serum albumin or a recombinant human albumin] and/or sodium chloride) in the pharmaceutical composition in addition to a botulinum neurotoxin active ingredient. A pharmaceutical composition is therefore a formulation which is suitable for diagnostic, therapeutic or cosmetic administration to a subject, such as a human patient. The pharmaceutical composition can be: in a lyophilized or vacuum dried condition, a solution formed after reconstitution of the lyophilized or vacuum dried pharmaceutical composition with saline or water, for example, or; as a solution that does not require reconstitution. As stated, a pharmaceutical composition can be liquid or solid. A pharmaceutical composition can be animal-protein free.

“Substantially free” means present at a level of less than one percent by weight of a culture medium, fermentation medium, pharmaceutical composition or other material in which the weight percent of a substance is assessed.

“Supplemental administration” as used herein refers to a botulinum administration that follows an initial neurotoxin administration.

“Therapeutic formulation” means a formulation that can be used to treat and thereby alleviate a disorder or a disease and/or symptom associated thereof, such as a disorder or a disease characterized by an activity of a peripheral muscle.

“Therapeutically effective amount” means the level, amount or concentration of an agent (e.g. such as a botulinum toxin or pharmaceutical composition comprising botulinum toxin) needed to treat a disease, disorder or condition without causing significant negative or adverse side effects.

“Toxin-naïve” means a patient who has not been administered a neurotoxin, for example a clostridial toxin.

“Treat,” “treating,” or “treatment” means an alleviation or a reduction (which includes some reduction, a significant reduction a near total reduction, and a total reduction), resolution or prevention (temporarily or permanently) of an disease, disorder or condition, so as to achieve a desired therapeutic or cosmetic result, such as by healing of injured or damaged tissue, or by altering, changing, enhancing, improving, ameliorating and/or beautifying an existing or perceived disease, disorder or condition.

“Unit” or “U” means an amount of active botulinum neurotoxin standardized to have equivalent neuromuscular blocking effect as a Unit of commercially available botulinum neurotoxin type A.

BoNT/A Fermentation

Disclosed embodiments comprise expansion of a starting cell line (growth and reproduction of Clostridium botulinum bacteria in a substantially APF culture medium), fermentation, harvest (removal of cellular debris) to provide a clarified, harvested culture that is then concentrated and diluted. Thus, in an embodiment the nine steps of the two column process comprise culturing, fermentation, harvest filtration, concentration, capture (anion) chromatography, polishing (cation) chromatography, buffer exchange, bioburden reduction and vial fill.

BoNT/E Fermentation

Gessler reported fermentation of BoNT/E as follows: the inoculum was grown in 100-mL flasks with 50 mL clostridial medium (CM) which consisted of 0.3% yeast extract, 0.75% meat extract, 0.75% peptone from casein, pancreatically digested, 0.75% peptone from meat, tryptically digested, 0.1% soluble starch, 0.5% d-glucose, 0.5% sodium chloride, 0.3% sodium acetate, 0.05% l-cysteine-HCl. The pH was adjusted to 7.0. The inoculum was incubated overnight at 26° C. in an anaerobic atmosphere (80% N₂, 5% H₂, 15% CO₂). A 10-L fermentor vessel with 1 L CM was inoculated and the pH initially adjusted to 6.8. To ensure an anaerobic atmosphere, the fermentor was supplied with a continuous N₂ overflow. If the bacteria grew well, 9 L of CM were added after ˜24 h to give a final culture volume of 10 L and incubated for another 4 days at 26° C. (See Frank Gessler, A new scaleable method for the purification of botulinum neurotoxin type E, J. Biotechnology 119 (2005) 204-211.) While BoNT/E producing C. botulinum can grow at temperatures from 4° C. to over 37° C., fastest growth is above 25° C.

In disclosed embodiments, fermentation is performed at, for example, between 5° C. and 20° C., less than 20° C., at 15° C., ±2° C., at 10° C., ±2° C., between 10° C. and 15° C., or the like.

Embodiments comprise maintaining a higher pH during the growth phase as compared to previous methods, which will maintain cell viability longer and result in increased duration of BoNT/E production. For example, disclosed embodiments comprise methods wherein the pH of the growth step is between 7.2 and 9.3, wherein the pH is 7.5±0.3, wherein the pH is 8.0±0.3, wherein the pH is 8.5±0.3, wherein the pH is 9.0±0.3, wherein the pH of the growth step is 7.5 or above, wherein the pH is 8.0 or above, or wherein the pH is 8.5 or above.

Master Cell Bank

Creation of a master cell bank is required for regulatory approval of a biologic. U.S. Pat. No. 8,324,349, incorporated entirely by reference, describes a method of making an animal product free (APF) master cell bank for a BoNT/A producing Clostridium botulinum.

Manufacturing

A number of steps are required to make a clostridial toxin pharmaceutical composition suitable for administration to a human or animal for a therapeutic, diagnostic, research or cosmetic purpose. These steps can comprise obtaining a purified clostridial toxin and then compounding the purified clostridial toxin. A first step can comprise plating and growing colonies of clostridial bacteria, typically on blood agar plates, in an environment conducive to anaerobic bacterial growth, such as in a warm anaerobic atmosphere. This step allows clostridial colonies with desirable morphology and other characteristics to be obtained. In a second step selected clostridial colonies can be fermented in a first suitable medium and if additionally desired, into a second fermentation medium. After a certain period of fermentation, the clostridial bacteria typically lyse and release clostridial toxin into the medium. Thirdly, the medium is purified so as to obtain a bulk toxin. Typically medium purification to obtain bulk toxin is carried out using, among other reagents, animal-derived enzymes, such as DNase and RNase, which are used to degrade and facilitate removal of nucleic acids. The resulting bulk toxin is a highly purified toxin with a particular specific activity. After stabilization in a suitable buffer, the bulk toxin can be compounded with one or more excipients to make a clostridial toxin pharmaceutical composition suitable for administration to a human. The clostridial toxin pharmaceutical composition can comprise a clostridial toxin as an active pharmaceutical ingredient (API). The pharmaceutical composition can also include one or more excipients, buffers, carriers, stabilizers, preservatives and/or bulking agents.

The clostridium toxin fermentation step can result in a fermentation medium solution that contains whole clostridium bacteria, lysed bacteria, culture medium nutrients and fermentation by-products. Filtration of this culture solution so as to remove gross elements, such as whole and lysed bacteria, provides a harvest/clarified medium. The clarified medium comprises a clostridial toxin and various impurities and is processed to obtain a concentrated clostridial toxin, which is called bulk toxin.

Fermentation and purification processes for obtaining a bulk clostridial toxin using one or more animal derived products (such as the milk digest casein, DNase and RNase) are known. An example of such a known non-animal product free (“NAPF”) process for obtaining a botulinum toxin complex is the Schantz process and modifications thereto. The Schantz process (from initial plating, cell culture through to fermentation and toxin purification) makes use of a number of products derived from animal sources such as, for example, animal derived Bacto Cooked Meat medium in the culture vial, Columbia Blood Agar plates for colony growth and selection, and casein in the fermentation media. Additionally, the Schantz bulk toxin purification process makes use of DNase and RNase from bovine sources to hydrolyze nucleic acids present in the toxin containing fermentation medium. Concerns have been expressed regarding a potential for a viral and transmissible spongiform encephalopathy (TSE), such as a bovine spongiform encephalopathy (BSE), contamination when animal products are used in a process for obtaining an API and/or in a process for making (compounding) a pharmaceutical composition using such an API.

Temperature is an important factor in fermentation, among other considerations. Gessler reported fermentation of BoNT/E as follows. The inoculum was grown in 100-mL flasks with 50 mL clostridial medium (CM) which consisted of 0.3% yeast extract, 0.75% meat extract, 0.75% peptone from casein, pancreatically digested, 0.75% peptone from meat, tryptically digested, 0.1% soluble starch, 0.5% d-glucose, 0.5% sodium chloride, 0.3% sodium acetate, 0.05% l-cysteine-HCl. The pH was adjusted to 7.0. The inoculum was incubated overnight at 26° C. in an anaerobic atmosphere (80% N₂, 5% H₂, 15% CO₂). A 10-L fermentor vessel with 1 L CM was inoculated and the pH initially adjusted to 6.8. To ensure an anaerobic atmosphere, the fermentor was supplied with a continuous N₂ overflow. If the bacteria grew well, 9 L of CM were added after ˜24 h to give a final culture volume of 10 L and incubated for another 4 days at 26° C. (See Frank Gessler, A new scaleable method for the purification of botulinum neurotoxin type E, J. BIOTECHNOLOGY 119 (2005) 204-211.) While BoNT/E producing C. botulinum can grow at temperatures from 4° C. to over 37° C., fastest growth is greater than 25° C. (See, e.g., Espelund et al., Botulism outbreaks in natural environments—an update, FRONTIERS IN MICROBIOLOGY (2014) doi: 10.3389/fmicb.2014.00287).

It is hypothesized by the inventors that fastest growth is not always correlated to optimal yield of active BoNT/E. For example, rapid growth of C. botulinum can result in a greater percentage of incorrectly folded and insoluble BoNT/E, decreasing yield. By decreasing temperature, and thus decreasing the growth rate, the percentage of insoluble protein will decrease and the percent yield will increase.

Chromatography, for example column chromatography, can be used to separate a particular protein (such as a botulinum neurotoxin) from a mixture of proteins, nucleic acids, cell debris, etc. in a process known as fractionation or purification. The protein mixture typically passes through a glass or plastic column containing, for example, a solid, often porous media (often referred to as beads or resin). Different proteins and other compounds pass through the matrix at different rates based on their specific chemical characteristics and the way in which these characteristics cause them to interact with the particular chromatographic media utilized.

The choice of media determines the type of chemical characteristic by which the fractionation of the proteins is based. There are four basic types of column chromatography; ion-exchange, gel filtration, affinity and hydrophobic interaction. Ion-exchange chromatography accomplishes fractionation based on surface electrostatic charge using a column packed with small beads carrying either a positive or a negative charge. In gel filtration chromatography, proteins are fractionated based on their size. In affinity chromatography, proteins are separated based on their ability to bind to specific chemical groups (ligand) attached to beads in the column matrix. Ligands can be biologically specific for a target protein. Hydrophobic interaction chromatography accomplishes fractionation based on surface hydrophobicity.

Neurotoxin Compositions

Embodiments disclosed herein comprise neurotoxin compositions, for example fast-acting neurotoxin compositions such as BoNT/E. Such neurotoxins can be formulated in any pharmaceutically acceptable formulation in any pharmaceutically acceptable form. The neurotoxin can also be used in any pharmaceutically acceptable form supplied by any manufacturer.

Embodiments disclosed herein comprise neurotoxin compositions, for example fast-recovery neurotoxins such as BoNT/E. Such neurotoxins can be formulated in any pharmaceutically acceptable formulation in any pharmaceutically acceptable form. The neurotoxin can also be used in any pharmaceutically acceptable form supplied by any manufacturer.

Embodiments disclosed herein can comprise multiple neurotoxins. For example, in embodiments disclosed compositions can comprise two types of neurotoxins, for example two types of botulinum neurotoxins, such as a fast-acting and a slower-acting neurotoxin, for example BoNT/E and BoNT/A. In embodiments, disclosed compositions can comprise a fragment of a botulinum neurotoxin, for example, a 50 kDa light chain (LC) fragment.

The neurotoxin can be made by a clostridial bacterium, such as by a Clostridium botulinum, Clostridium butyricum, or Clostridium beratti bacterium. Additionally, the neurotoxin can be a modified neurotoxin; that is a neurotoxin that has at least one of its amino acids deleted, modified or replaced, as compared to the native or wild type neurotoxin. Furthermore, the neurotoxin can be a recombinantly produced neurotoxin or a derivative or fragment thereof.

In embodiments, a disclosed BoNT/E composition has 40% amino acid homology compared with type A and they share the same basic domain structure consisting of 2 chains, a 100 kDa heavy chain (HC) and a 50 kDa light chain (LC), linked by a disulfide bond (Whelan 1992). The HC contains the receptor binding domain and the translocation domain while the LC contains the synaptosomal-associated protein (SNAP) enzymatic activity. The domain structure is the same structure shared by all botulinum neurotoxin serotypes.

In disclosed embodiments, the neurotoxin is formulated in unit dosage form; for example, it can be provided as a sterile solution in a vial or as a vial or sachet containing a lyophilized powder for reconstituting a suitable vehicle such as saline for injection.

In embodiments, the botulinum toxin is formulated in a solution containing saline and pasteurized human serum albumin (HSA), which stabilizes the toxin and minimizes loss through non-specific adsorption. The solution can comprise a buffer, for example a buffer with a PKa value between 6.0 and 8.0, high water solubility, and minimal organic solubility, such as, for example, phosphate buffer, and other suitable types. The solution can be sterile filtered (0.2μ filter), filled into individual vials and then vacuum-dried to give a sterile lyophilized powder. In use, the powder can be reconstituted by the addition of sterile unpreserved normal saline (sodium chloride 0.9% for injection).

In an embodiment, BoNT/E is supplied in a sterile solution for injection with a 5-mL vial nominal concentration of 50 ng/mL in 0.03 M sodium phosphate, 0.12 M sodium chloride, and 1 mg/mL HSA, at pH 6.0.

In an embodiment, BoNT/E is supplied in a sterile solution for injection with a 5-mL vial nominal concentration of 35 ng/mL in 0.03 M sodium phosphate, 0.12 M sodium chloride, and 1 mg/mL HSA, at pH 6.0.

In an embodiment, BoNT/E is supplied in a sterile solution for injection with a 5-mL vial nominal concentration of 30 ng/mL in 0.03 M sodium phosphate, 0.12 M sodium chloride, and 1 mg/mL HSA, at pH 6.0.

In an embodiment, BoNT/E is supplied in a sterile solution for injection with a 5-mL vial nominal concentration of 20 ng/mL in 0.03 M sodium phosphate, 0.12 M sodium chloride, and 1 mg/mL HSA, at pH 6.0.

In an embodiment, BoNT/E is supplied in a sterile solution for injection with a 5-mL vial nominal concentration of 10 ng/mL in 0.03 M sodium phosphate, 0.12 M sodium chloride, and 1 mg/mL HSA, at pH 6.0.

In an embodiment, BoNT/E is supplied in a sterile solution for injection with a 5-mL vial nominal concentration of 5 ng/mL in 0.03 M sodium phosphate, 0.12 M sodium chloride, and 1 mg/mL HSA, at pH 6.0.

In an embodiment, BoNT/E is supplied in a sterile solution for injection with a 5-mL vial nominal concentration of 1 ng/mL in 0.03 M sodium phosphate, 0.12 M sodium chloride, and 1 mg/mL HSA, at pH 6.0.

Although the composition may only contain a single type of neurotoxin, for example BoNT/E, disclosed compositions can include two or more types of neurotoxins, which can provide enhanced therapeutic effects of the disorders. For example, a composition administered to a patient can include BoNT/A and BoNT/E. Administering a single composition containing two different neurotoxins can permit the effective concentration of each of the neurotoxins to be lower than if a single neurotoxin is administered to the patient while still achieving the desired therapeutic effects.

Methods of Use

Methods disclosed herein can comprise administration of a fast-acting neurotoxin to a patient, for example a patient suffering from, for example, hyperhidrosis, or from glabellar wrinkles. In a preferred embodiment the neurotoxin is BoNT/E, for example wild-type BoNT/E.

Embodiments comprise use of disclosed compositions and methods in conjunction with a surgical procedure. For example, disclosed embodiments can comprise neurotoxin treatments performed in conjunction with, for example cosmetic procedures such as glabellar line treatment, therapeutic procedures such as migraine treatment, or the like.

Disclosed fast-acting neurotoxin compositions can be administered using, for example, a needle or a needleless device. In certain embodiments, the method comprises subdermally injecting the composition in the individual. For example, administration may comprise injecting the composition through a needle, for example about 30 gauge. In embodiments, the method comprises administering a composition comprising BoNT/E.

Injection of the compositions can be carried out by syringe, catheters, needles and other appropriate means. The injection can be performed on any area of the mammal's body that is in need of treatment, including, but not limited to, face, neck, torso, arms, hands, legs, and feet. The injection can be into any position in the specific area such as epidermis, dermis, fat, muscle, or subcutaneous layer.

Administration of disclosed compositions can comprise administration, for example, injection, into or in the vicinity of one or more of the following skeletal muscles, for example, the occipitofrontalis, nasalis, orbicularis oris, depressor anguli oris, platysma, sternohyoid, serratus anterior, rectus abdominis, external oblique, tensor fasciae latae, brachioradialis, Iliacus, psoas major, pectineus, adductor longus, sartorius, gracillis, vastus lateralis, rectus femoris, vastus medialis, tendon of quadriceps femoris, patella, gastroctnemius, soleus, tibia, fibularis longus, tibialis anterior, patellar ligament, iliotibial tract, hypothenar muscles, thenar muscles, flexor carpi ulnaris, flexor digitorum superficialis, palmaris longus, flexor carpi radials, brachioradialis, pronator teres, brachialis, biceps brachii, triceps brachii, pectoralis major, deltoid, trapezius, sternocleidomastoid, masseter, orbicularis oculi, temporalis, epicranial aponeurosis, teres major, extensor digitorum, extensor carpi ulnaris, anconeus, abductor policis longus, plantaris, calcanel tendon, soleus, adductor magnus, gluteus maximas, gluteus medius, latissimus dorsi, intraspinatus, and combinations thereof, and the like.

Administration of disclosed compositions can comprise, for example, administration, for example injection, into or in the vicinity of one or more of the following nerves, for example, the axillary nerve, phrenic nerve, spinal ganglion, spinal cord, sympathetic ganglia chain, pudendal nerve, common palmar digital nerve, ulnar nerve, deep branch of the ulnar nerve, sciatic nerve, peroneal nerve, tibial nerve, saphenous nerve, interosseous nerve, superficial peroneal nerve, intermediate dorsal cutaneous nerve, medial plantar nerve, medial dorsal cutaneous nerve, deep peroneal nerve, muscular branches of tibial nerve, intrapatellar branch of saphenous nerve, common peroneal nerve, muscular branch of femoral nerve, anterior cutaneous branches of femoral nerve, muscular branches of sciatic nerve, femoral nerve, iliolinguinal, filum terminate, iliohypogastric, obturator, ulnar, radial, obturator, radial, subcostal, intercostal, dorsal branches of the intercostal, medial cutaneous branches of the intercostal, musculaneous, deltoid, vagus, brachial plexus, supraclavicular, facial, auriculotemporal, combinations thereof, and the like.

The frequency and the amount of the injection for each particular case can be determined by the person of ordinary skill in the art. For example, injections to the axilla are employed in treating axillary hyperhidrosis.

Although examples of routes of administration and dosages are provided, the appropriate route of administration and dosage are generally determined on a case by case basis by the attending physician. Such determinations are routine to one of ordinary skill in the art. For example, the route and dosage for administration of a Clostridial neurotoxin according to the present disclosed invention can be selected based upon criteria such as the solubility characteristics of the neurotoxin chosen as well as the intensity and scope of the cosmetic condition being treated.

In embodiments, administration can comprise one or more injections, for example injections substantially along an incision site or line or lines, or around the perimeter of a lesion. In embodiments, administration can comprise injections in a specific pattern, for example, a W pattern, and X patter, a Z pattern, a star pattern, a circle pattern, a half circle pattern, a square pattern, a rectangle pattern, a line pattern, a crescent patter, a perimeter pattern, a spiral pattern, or combinations thereof. In embodiments, injection sites can be marked, for example with a pen or marker, prior to injection.

Methods disclosed herein can comprise administration of a neurotoxin, for example a fast-acting neurotoxin, to a patient, wherein the dosage of the neurotoxin is expressed in protein amount, for example protein amount per administration, for example nanograms (ng). In an embodiment the fast-acting neurotoxin is a botulinum toxin, for example BoNT/E.

In embodiments, the dose of the neurotoxin is expressed in protein amount or concentration. For example, in embodiments the neurotoxin, for example BoNT/E, can be administered in an amount of between about 0.2 ng and 20 ng. In an embodiment, the neurotoxin is administered in an amount of between about 0.3 ng and 19 ng, about 0.4 ng and 18 ng, about 0.5 ng and 17 ng, about 0.6 ng and 16 ng, about 0.7 ng and 15 ng, about 0.8 ng and 14 ng, about 0.9 ng and 13 ng, about 1.0 ng and 12 ng, about 1.5 ng and 11 ng, about 2 ng and 10 ng, about 5 ng and 7 ng, and the like, into a target tissue such as a muscle.

In embodiments, administration can comprise a total dose of between 5 and 7 ng, between 7 and 9 ng, between 9 and 11 ng, between 11 and 13 ng, between 13 and 15 ng, between 15 and 17 ng, between 17 and 19 ng, or the like.

In embodiments, administration can comprise a total dose of not more than 5 ng, not more than 6 ng, not more than 7 ng, not more than 8 ng, not more than 9 ng, not more than 10 ng, not more than 11 ng, not more than 12 ng, not more than 13 ng, not more than 14 ng, not more than 15 ng, not more than 16 ng, not more than 17 ng, not more than 18 ng, not more than 19 ng, not more than 20 ng, or the like.

In embodiments, administration can comprise a total dose of not less than 5 ng, not less than 6 ng, not less than 7 ng, not less than 8 ng, not less than 9 ng, not less than 10 ng, not less than 11 ng, not less than 12 ng, not less than 13 ng, not less than 14 ng, not less than 15 ng, not less than 16 ng, not less than 17 ng, not less than 18 ng, not less than 19 ng, not less than 20 ng, or the like.

In embodiments, administration can comprise a total dose of about 0.1 ng of a neurotoxin, for example BoNT/E, 0.2 ng of a neurotoxin, 0.3 ng of a neurotoxin, 0.4 ng of a neurotoxin, 0.5 ng of a neurotoxin, 0.6 ng of a neurotoxin, 0.7 ng of a neurotoxin, 0.8 ng of a neurotoxin, 0.9 ng of a neurotoxin, 1.0 ng of a neurotoxin, 1.1 ng of a neurotoxin, 1.2 ng of a neurotoxin, 1.3 ng of a neurotoxin, 1.4 ng of a neurotoxin, 1.5 ng of a neurotoxin, 1.6 ng of a neurotoxin, 1.7 ng of a neurotoxin, 1.8 ng of a neurotoxin, 1.9 ng of a neurotoxin, 2.0 ng of a neurotoxin, 2.1 ng of a neurotoxin, 2.2 ng of a neurotoxin, 2.3 ng of a neurotoxin, 2.4 ng of a neurotoxin, 2.5 ng of a neurotoxin, 2.6 ng of a neurotoxin, 2.7 ng of a neurotoxin, 2.8 ng of a neurotoxin, 2.9 ng of a neurotoxin, 3.0 ng of a neurotoxin, 3.1 ng of a neurotoxin, 3.2 ng of a neurotoxin, 3.3 ng of a neurotoxin, 3.4 ng of a neurotoxin, 3.5 ng of a neurotoxin, 3.6 ng of a neurotoxin, 3.7 ng of a neurotoxin, 3.8 ng of a neurotoxin, 3.9 ng of a neurotoxin, 4.0 ng of a neurotoxin, 4.1 ng of a neurotoxin, 4.2 ng of a neurotoxin, 4.3 ng of a neurotoxin, 4.4 ng of a neurotoxin, 4.5 ng of a neurotoxin, 5 ng of a neurotoxin, 6 ng of a neurotoxin, 7 ng of a neurotoxin, 8 ng of a neurotoxin, 9 ng of a neurotoxin, 10 ng of a neurotoxin, 11 ng of a neurotoxin, 12 ng of a neurotoxin, 13 ng of a neurotoxin, 14 ng of a neurotoxin, 15 ng of a neurotoxin, 16 ng of a neurotoxin, 17 ng of a neurotoxin, 18 ng of a neurotoxin, 19 ng of a neurotoxin, 20 ng of a neurotoxin, or the like.

In embodiments, administration can comprise a dose per injection of, for example, about 0.1 ng of a BoNT/E, 0.2 ng of a BoNT/E, 0.3 ng of a BoNT/E, 0.4 ng of a BoNT/E, 0.5 ng of a BoNT/E, 0.6 ng of a BoNT/E, 0.7 ng of a BoNT/E, 0.8 ng of a BoNT/E, 0.9 ng of a BoNT/E, 1.0 ng of a BoNT/E, 1.1 ng of a BoNT/E, 1.2 ng of a BoNT/E, 1.3 ng of a BoNT/E, 1.4 ng of a BoNT/E, 1.5 ng of a BoNT/E, 1.6 ng of a BoNT/E, 1.7 ng of a BoNT/E, 1.8 ng of a BoNT/E, 1.9 ng of a BoNT/E, 2.0 ng of a BoNT/E, 2.1 ng of a BoNT/E, 2.2 ng of a BoNT/E, 2.3 ng of a BoNT/E, 2.4 ng of a BoNT/E, 2.5 ng of a BoNT/E, 2.6 ng of a BoNT/E, 2.7 ng of a BoNT/E, 2.8 ng of a BoNT/E, 2.9 ng of a BoNT/E, 3.0 ng of a BoNT/E, 3.1 ng of a BoNT/E, 3.2 ng of a BoNT/E, 3.3 ng of a BoNT/E, 3.4 ng of a BoNT/E, 3.5 ng of a BoNT/E, 3.6 ng of a BoNT/E, 3.7 ng of a BoNT/E, 3.8 ng of a BoNT/E, 3.9 ng of a BoNT/E, 4.0 ng of a BoNT/E, 4.1 ng of a BoNT/E, 4.2 ng of a BoNT/E, 4.3 ng of a BoNT/E, 4.4 ng of a BoNT/E, 4.5 ng of a BoNT/E, 5 ng of a BoNT/E, 6 ng of a BoNT/E, 7 ng of a BoNT/E, 8 ng of a BoNT/E, 9 ng of a BoNT/E, 10 ng of a BoNT/E, or the like.

In embodiments, administration can comprise a dose per injection of about 0.1 ng of a neurotoxin, for example BoNT/E, 0.2 ng of a neurotoxin, 0.3 ng of a neurotoxin, 0.4 ng of a neurotoxin, 0.5 ng of a neurotoxin, 0.6 ng of a neurotoxin, 0.7 ng of a neurotoxin, 0.8 ng of a neurotoxin, 0.9 ng of a neurotoxin, 1.0 ng of a neurotoxin, 1.1 ng of a neurotoxin, 1.2 ng of a neurotoxin, 1.3 ng of a neurotoxin, 1.4 ng of a neurotoxin, 1.5 ng of a neurotoxin, 1.6 ng of a neurotoxin, 1.7 ng of a neurotoxin, 1.8 ng of a neurotoxin, 1.9 ng of a neurotoxin, 2.0 ng of a neurotoxin, 2.1 ng of a neurotoxin, 2.2 ng of a neurotoxin, 2.3 ng of a neurotoxin, 2.4 ng of a neurotoxin, 2.5 ng of a neurotoxin, 2.6 ng of a neurotoxin, 2.7 ng of a neurotoxin, 2.8 ng of a neurotoxin, 2.9 ng of a neurotoxin, 3.0 ng of a neurotoxin, 3.1 ng of a neurotoxin, 3.2 ng of a neurotoxin, 3.3 ng of a neurotoxin, 3.4 ng of a neurotoxin, 3.5 ng of a neurotoxin, 3.6 ng of a neurotoxin, 3.7 ng of a neurotoxin, 3.8 ng of a neurotoxin, 3.9 ng of a neurotoxin, 4.0 ng of a neurotoxin, 4.1 ng of a neurotoxin, 4.2 ng of a neurotoxin, 4.3 ng of a neurotoxin, 4.4 ng of a neurotoxin, 4.5 ng of a neurotoxin, 5 ng of a neurotoxin, 6 ng of a neurotoxin, 7 ng of a neurotoxin, 8 ng of a neurotoxin, 9 ng of a neurotoxin, 10 ng of a neurotoxin, or the like.

In embodiments, the total cumulative dose of neurotoxin administered is tracked and recorded.

The fast-acting neurotoxin, for example a BoNT/E, can be administered in an amount of between about 10⁻³ U/kg and about 35 U/kg patient body weight. In an embodiment, the neurotoxin is administered in an amount of between about 10⁻² U/kg and about 25 U/kg. In another embodiment, the neurotoxin is administered in an amount of between about 10⁻¹ U/kg and about 15 U/kg. In another embodiment, the neurotoxin is administered in an amount of between about 1 U/kg and about 10 U/kg. In many instances, an administration of from about 1 unit to about 500 units of a neurotoxin, such as a BoNT/E, provides effective therapeutic relief. In an embodiment, from about 5 units to about 200 units of a neurotoxin, such as a BoNT/E, can be used and in another embodiment, from about 10 units to about 100 units of a neurotoxin, such as a BoNT/E, can be locally administered into a target tissue such as a muscle.

In embodiments, administration can comprise a dose of about 2 units of a neurotoxin, for example a BoNT/E, or about 3 units of a neurotoxin, or about 4 units of a neurotoxin, or about 5 units of a neurotoxin, or about 6 units of a neurotoxin, or about 7 units of a neurotoxin, or about 8 units of a neurotoxin, or about 9 units of a neurotoxin, or about 10 units of a neurotoxin, or about 15 units of a neurotoxin, or about 20 units of a neurotoxin, or about 30 units of a neurotoxin, or about 40 units of a neurotoxin, or about 50 units of a neurotoxin, or about 60 units of a neurotoxin, or about 70 units of a neurotoxin, or about 80 units of a neurotoxin, or about 90 units of a neurotoxin, or about 100 units of a neurotoxin, or about 110 units of a neurotoxin, or about 120 units of a neurotoxin, or about 130 units of a neurotoxin, or about 140 units of a neurotoxin, or about 150 units of a neurotoxin, or about 160 units of a neurotoxin, or about 170 units of a neurotoxin, or about 180 units of a neurotoxin, or about 190 units of a neurotoxin, or about 200 units of a neurotoxin, or about 210 units of a neurotoxin, or about 220 units of a neurotoxin, or about 230 units of a neurotoxin, or about 240 units of a neurotoxin, or about 250 units of a neurotoxin, or about 260 units of a neurotoxin, or about 270 units of a neurotoxin, or about 280 units of a neurotoxin, or about 290 units of a neurotoxin, or about 290 units of a neurotoxin, or about 300 units of a neurotoxin, or about 310 units of a neurotoxin, or about 320 units of a neurotoxin, or about 330 units of a neurotoxin, or about 340 units of a neurotoxin, or about 350 units of a neurotoxin, or about 360 units of a neurotoxin, or about 370 units of a neurotoxin, or about 380 units of a neurotoxin, or about 390 units of a neurotoxin, or about 400 units of a neurotoxin, or about 410 units of a neurotoxin, or about 420 units of a neurotoxin, or about 430 units of a neurotoxin, or about 440 units of a neurotoxin, or about 450 units of a neurotoxin, or about 460 units of a neurotoxin, or about 470 units of a neurotoxin, or about 480 units of a neurotoxin, or about 490 units of a neurotoxin, or about 500 units of a neurotoxin, or the like.

In embodiments, administration can comprise a dose of about 4 units of a BoNT/E, or about 5 units of a BoNT/E, or about 6 units of a BoNT/E, or about 7 units of a BoNT/E, or about 8 units of a BoNT/E, or about 10 units of a BoNT/E, or about 15 units of a BoNT/E, or about 20 units of a BoNT/E, or about 30 units of a BoNT/E, or about 40 units of a BoNT/E, or about 50 units of a BoNT/E, or about 60 units of a BoNT/E, or about 70 units of a BoNT/E, or about 80 units of a BoNT/E, or about 90 units of a BoNT/E, or about 100 units of a BoNT/E, or about 110 units of a BoNT/E, or about 120 units of a BoNT/E, or about 130 units of a BoNT/E, or about 140 units of a BoNT/E, or about 150 units of a BoNT/E, or about 160 units of a BoNT/E, or about 170 units of a BoNT/E, or about 180 units of a BoNT/E, or about 190 units of a BoNT/E, or about 200 units of a BoNT/E, or about 210 units of a BoNT/E, or about 220 units of a BoNT/E, or about 230 units of a BoNT/E, or about 240 units of a BoNT/E, or about 250 units of a BoNT/E, or about 260 units of a BoNT/E, or about 270 units of a BoNT/E, or about 280 units of a BoNT/E, or about 290 units of a BoNT/E, or about 290 units of a BoNT/E, or about 300 units of a BoNT/E, or about 310 units of a BoNT/E, or about 320 units of a BoNT/E, or about 330 units of a BoNT/E, or about 340 units of a BoNT/E, or about 350 units of a neurotoxin, or about 360 units of a BoNT/E, or about 370 units of a BoNT/E, or about 380 units of a BoNT/E, or about 390 units of a BoNT/E, or about 400 units of a BoNT/E, or about 410 units of a BoNT/E, or about 420 units of a BoNT/E, or about 430 units of a BoNT/E, or about 440 units of a BoNT/E, or about 450 units of a BoNT/E, or about 460 units of a BoNT/E, or about 470 units of a BoNT/E, or about 480 units of a BoNT/E, or about 490 units of a BoNT/E, or about 500 units of a BoNT/E, or the like.

In embodiments, administration of the neurotoxin, for example a clostridial neurotoxin such as a BoNT/E, can be repeated after a time interval of, for example, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 32 days, at least 33 days, at least 34 days, at least 35 days, at least 36 days, at least 37 days, at least 38 days, at least 39 days, at least 40 days, at least 41 days, at least 42 days, at least 43 days, at least 44 days, at least 45 days, at least 46 days, at least 47 days, at least 48 days, at least 49 days, at least 50 days, at least 51 days, at least 52 days, at least 53 days, at least 54 days, at least 55 days, at least 56 days, at least 57 days, at least 58 days, at least 59 days, at least 60 days, or the like.

In embodiments, administration of the neurotoxin, for example a BoNT/E, can be repeated after a time interval of, for example, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, or the like.

In embodiments, administration of the neurotoxin, for example the BoNT/E, can be repeated after a time interval of, for example, not more than 4 weeks, not more than 5 weeks, not more than 6 weeks, not more than 7 weeks, not more than 8 weeks, not more than 9 weeks, not more than 10 weeks, not more than 11 weeks, not more than 12 weeks, not more than 13 weeks, not more than 14 weeks, not more than 15 weeks, not more than 16 weeks, or the like.

Ultimately, however, both the quantity of toxin administered and the frequency of its administration will be at the discretion of the physician responsible for the treatment and will be commensurate with questions of safety and the effects produced by the toxin.

In embodiments, administration of the fast-acting neurotoxin, for example BoNT/E, is performed after a surgical procedure. For example, administration can be performed, within 1 minute after the procedure, within 2 minutes after the procedure, within 3 minutes after the procedure, within 4 minutes after the procedure, within 5 minutes after the procedure, within 6 minutes after the procedure, within 7 minutes after the procedure, within 8 minutes after the procedure, within 9 minutes after the procedure, within 10 minutes after the procedure, within 20 minutes after the procedure, within 30 minutes after the procedure, within 40 minutes after the procedure, within 50 minutes after the procedure, within 60 minutes after the procedure, within 90 minutes after the procedure, within 120 minutes after the procedure, within 180 minutes after the procedure, within 240 minutes after the procedure, within 300 minutes after the procedure, or the like.

In embodiments, administration of a fast-acting neurotoxin, for example BoNT/E, is performed after a surgical procedure. For example, administration can be performed, within 1 minute or less after the procedure, within 2 minutes or less after the procedure, within 3 minutes or less after the procedure, within 4 minutes or less after the procedure, within 5 minutes or less after the procedure, within 6 minutes or less after the procedure, within 7 minutes or less after the procedure, within 8 minutes or less after the procedure, within 9 minutes or less after the procedure, within 10 minutes or less after the procedure, within 20 or less minutes after the procedure, within 30 minutes or less after the procedure, within 40 minutes or less after the procedure, within 50 minutes or less after the procedure, within 60 minutes or less after the procedure, within 90 minutes or less after the procedure, within 120 minutes or less after the procedure, within 180 minutes or less after the procedure, within 240 minutes or less after the procedure, within 300 minutes or less after the procedure, or the like.

In embodiments, administration of the fast acting neurotoxin, for example BoNT/E, is performed prior to a surgical procedure. In embodiments, the administration is performed, for example, within 36 hours before the procedure, within 24 hours before the procedure, within 22 hours before the procedure, within 20 hours before the procedure, within 18 hours before the procedure, within 16 hours before the procedure, within 14 hours before the procedure, within 12 hours before the procedure, within 11 hours before the procedure, within 10 hours before the procedure, within 9 hours before the procedure, within 8 hours before the procedure, within 7 hours before the procedure, within 6 hours before the procedure, within 5 hours before the procedure, within 4 hours before the procedure, within 3 hours before the procedure, within 2 hours before the procedure, within 60 minutes before the procedure, within 50 minutes before the procedure, within 40 minutes before the procedure, within 30 minutes before the procedure, within 20 minutes before the procedure, within 10 minutes before the procedure, within 5 minutes before the procedure, within 2 minutes before the procedure, or the like.

In embodiments, administration of the fast acting neurotoxin, for example BoNT/E, is performed prior to a surgical procedure. In embodiments, the administration is performed, for example, not less than 48 hours before the procedure, not less than 36 hours before the procedure, not less than 24 hours before the procedure, not less than 22 hours before the procedure, not less than 20 hours before the procedure, not less than 18 hours before the procedure, not less than 16 hours before the procedure, not less than 14 hours before the procedure, not less than 12 hours before the procedure, not less than 11 hours before the procedure, not less than 10 hours before the procedure, not less than 9 hours before the procedure, not less than 8 hours before the procedure, not less than 7 hours before the procedure, not less than 6 hours before the procedure, not less than 5 hours before the procedure, not less than 4 hours before the procedure, not less than 3 hours before the procedure, not less than 2 hours before the procedure, not less than 60 minutes before the procedure, not less than 50 minutes before the procedure, not less than 40 minutes before the procedure, not less than 30 minutes before the procedure, not less than 20 minutes before the procedure, not less than 10 minutes before the procedure, not less than 5 minutes before the procedure, not less than 2 minutes before the procedure, or the like.

In embodiments, administration of the fast acting neurotoxin, for example BoNT/E, is performed concurrently with a surgical procedure.

In embodiments, administration of the fast acting neurotoxin, for example BoNT/E, is performed after a surgical procedure. For example, administration can be performed, within 1 minute after the procedure, within 2 minutes after the procedure, within 3 minutes after the procedure, within 4 minutes after the procedure, within 5 minutes after the procedure, within 6 minutes after the procedure, within 7 minutes after the procedure, within 8 minutes after the procedure, within 9 minutes after the procedure, within 10 minutes after the procedure, within 20 minutes after the procedure, within 30 minutes after the procedure, within 40 minutes after the procedure, within 50 minutes after the procedure, within 60 minutes after the procedure, within 90 minutes after the procedure, within 2 hours after the procedure, within 3 hours after the procedure, within 4 hours after the procedure, within 5 hours after the procedure, within 6 hours after the procedure, within 7 hours after the procedure, within 8 hours after the procedure, within 9 hours after the procedure, within 10 hours after the procedure, within 11 hours after the procedure, within 12 hours after the procedure, within 16 hours after the procedure, or the like.

Before administering compositions disclosed herein, careful consideration is given to the anatomy of the treatment site. For example, in embodiments, the therapeutic goal is to inject the area with the highest concentration of neuromuscular junctions, if known. For example, in the case of intramuscular administration, before injecting the muscle the position of the needle in the muscle can be confirmed by putting the muscle through its range of motion and observing the resultant motion of the needle end. General anesthesia, local anesthesia and sedation are used according to the age of the patient, the number of sites to be injected, and the particular needs of the patient. More than one injection and/or sites of injection may be necessary to achieve the desired result. Also, some injections, depending on the muscle to be injected, may require the use of fine, hollow, TEFLON®-coated needles, guided by electromyography.

Administration sites useful for practicing disclosed embodiments can comprise any area where muscle and/or nerve activity is to be reduced. For example, administration can be made in the area of a traumatic injury.

The frequency and the amount of injection under the disclosed methods can be determined based on the nature and location of the particular area being treated. In certain cases, however, repeated or supplemental injections may be desired to achieve optimal results. The frequency and the amount of the injection for each particular case can be determined by the person of ordinary skill in the art.

Methods disclosed herein can comprise supplemental administration of a fast-acting neurotoxin, for example BoNT/E, to a patient after an initial administration. Embodiments comprising supplemental administration can further comprise doctor or patient evaluation of the results of a prior neurotoxin administration. Such evaluation can comprise the use of, for example, photographs, scanning, or the like.

In embodiments, evaluation of the results of the initial neurotoxin administration can be performed within, for example, 6 hours of the initial administration, 8 hours of the initial administration, 10 hours of the initial administration, 12 hours of the initial administration, 14 hours of the initial administration, 16 hours of the initial administration, 18 hours of the initial administration, 24 hours of the initial administration, 30 hours of the initial administration, 36 hours of the initial administration, 42 hours of the initial administration, 48 hours of the initial administration, 54 hours of the initial administration, 60 hours of the initial administration, 66 hours of the initial administration, 72 hours of the initial administration, 78 hours of the initial administration, 84 hours of the initial administration, 90 hours of the initial administration, 96 hours of the initial administration, 102 hours of the initial administration, 108 hours of the initial administration, 114 hours of the initial administration, 120 hours of the initial administration, 1 week of the initial administration, 2 weeks of the initial administration, 3 weeks of the initial administration, 4 weeks of the initial administration, 5 weeks of the initial administration, 6 weeks of the initial administration, 7 weeks of the initial administration, 8 weeks of the initial administration, 9 weeks of the initial administration, 10 weeks of the initial administration, 11 weeks of the initial administration, 12 weeks of the initial administration, or the like.

In embodiments comprising a supplemental administration, administration of the supplemental dose can be performed, within, for example, 6 hours of the evaluation, 8 hours of the evaluation, 10 hours of the evaluation, 12 hours of the evaluation, 14 hours of the evaluation, 16 hours of the evaluation, 18 hours of the evaluation, 24 hours of the evaluation, 30 hours of the evaluation, 36 hours of the evaluation, 42 hours of the evaluation, 48 hours of the evaluation, 54 hours of the evaluation, 60 hours of the evaluation, 66 hours of the evaluation, 72 hours of the evaluation, 78 hours of the evaluation, 84 hours of the evaluation, 90 hours of the evaluation, 96 hours of the evaluation, 102 hours of the evaluation, 108 hours of the evaluation, 114 hours of the evaluation, 120 hours of the evaluation, 1 week of the evaluation, 2 weeks of the evaluation, 3 weeks of the evaluation, 4 weeks of the evaluation, 5 weeks of the evaluation, 6 weeks of the evaluation, 7 weeks of the evaluation, 8 weeks of the evaluation, 9 weeks of the evaluation, 10 weeks of the evaluation, 11 weeks of the evaluation, 12 weeks of the evaluation, or the like.

In embodiments, the supplemental administration can be performed, for example, within, for example, 6 hours of the initial administration, 8 hours of the initial administration, 10 hours of the initial administration, 12 hours of the initial administration, 14 hours of the initial administration, 16 hours of the initial administration, 18 hours of the initial administration, 24 hours of the initial administration, 30 hours of the initial administration, 36 hours of the initial administration, 42 hours of the initial administration, 48 hours of the initial administration, 54 hours of the initial administration, 60 hours of the initial administration, 66 hours of the initial administration, 72 hours of the initial administration, 78 hours of the initial administration, 84 hours of the initial administration, 90 hours of the initial administration, 96 hours of the initial administration, 102 hours of the initial administration, 108 hours of the initial administration, 114 hours of the initial administration, 120 hours of the initial administration, 1 week of the initial administration, 2 weeks of the initial administration, 3 weeks of the initial administration, 4 weeks of the initial administration, 5 weeks of the initial administration, 6 weeks of the initial administration, 7 weeks of the initial administration, 8 weeks of the initial administration, 9 weeks of the initial administration, 10 weeks of the initial administration, 11 weeks of the initial administration, 12 weeks of the initial administration, or the like.

Methods disclosed herein can provide rapid-onset effects, for example, using a fast-acting neurotoxin such as a BoNT/E. For example, disclosed embodiments can provide effect within, for example, 30 minutes after administration of the fast-acting neurotoxin, 45 minutes after administration, 60 minutes after administration, 75 minutes after administration, 90 minutes after administration, 2 hours after administration, 3 hours after administration, 4 hours after administration, 5 hours after administration, 6 hours after administration, 7 hours after administration, 8 hours after administration, 9 hours after administration, 10 hours after administration, 11 hours after administration, 12 hours after administration, 13 hours after administration, 14 hours after administration, 15 hours after administration, 16 hours after administration, 17 hours after administration, 18 hours after administration, 19 hours after administration, 20 hours after administration, 21 hours after administration, 22 hours after administration, 23 hours after administration, 24 hours after administration, 30 hours after administration, 36 hours after administration, 42 hours after administration, 48 hours after administration, 3 days after administration, 4 days after administration, 5 days after administration, 6 days after administration, 7 days after administration, 8 days after administration, 9 days after administration, 10 days after administration, 11 days after administration, 12 days after administration, or the like.

Methods disclosed herein can provide effects of a shorter direction, for example, using a fast-recovery neurotoxin. For example, disclosed embodiments can provide effects that subside within, for example, 3 days after administration, 4 days after administration, 5 days after administration, 6 days after administration, 7 days after administration, 8 days after administration, 9 days after administration, 10 days after administration, 11 days after administration, 12 days after administration, 13 days after administration, 14 days after administration, 15 days after administration, 16 days after administration, 17 days after administration, 18 days after administration, 19 days after administration, 20 days after administration, 21 days after administration, 22 days after administration, 23 days after administration, 24 days after administration, 25 days after administration, 26 days after administration, 27 days after administration, 28 days after administration, 29 days after administration, 30 days after administration, 45 days after administration, 60 days after administration, 75 days after administration, 90 days after administration, 105 days after administration, or the like.

Side-effects can be associated with botulinum injections. Disclosed embodiments can provide neurotoxin treatments that result in fewer side effects, or side effects of a shorted duration, than conventional neurotoxin treatments. For example, disclosed embodiments can result in fewer (or shorter duration) instances of double vision or blurred vision, eyelid paralysis (subject cannot lift eyelid all the way open), loss of facial muscle movement, hoarseness, loss of bladder control, shortness of breath, difficulty in swallowing, difficulty speaking, death, and the like.

Further, disclosed embodiments can provide patients with effects of a more-certain duration. For example, with a longer acting neurotoxin, a 20% variance in duration of effects can result in a month's difference in effective duration. With the disclosed fast-recovery neurotoxins, this 20% variance produces a much less drastic difference in effective duration.

Supplemental administrations of a fast-acting neurotoxin can effectively modify or augment previous cosmetic neurotoxin administrations. For example, methods disclosed herein can comprise a supplemental administration to correct an unsatisfactory result from a previous administration, or to increase the effects of a previous administration, or to accelerate the onset of results as compared to those achieved using non fast-acting neurotoxins.

A kit for practicing disclosed embodiments is also encompassed by the present disclosure. The kit can comprise a 30 gauge or smaller needle and a corresponding syringe. The kit also comprises a Clostridial neurotoxin composition, such as a BoNT/E toxin composition. The neurotoxin composition may be provided in the syringe. The composition is injectable through the needle. The kits are designed in various forms based the sizes of the syringe and the needles and the volume of the injectable composition contained therein, which in turn are based on the specific cosmetic deficiencies the kits are designed to treat.

### EXAMPLES

The following non-limiting examples are provided for illustrative purposes only in order to facilitate a more complete understanding of representative embodiments. This example should not be construed to limit any of the embodiments described in the present specification.

### Example 1

### BoNT/A Fermentation

A starting cell line was expanded (growth and reproduction of Clostridium botulinum bacteria in a substantially APF culture medium, fermentation, harvest, removal of cellular debris) to provide a clarified, harvested culture that is then concentrated and diluted. Thus, in an embodiment the nine steps of the two column process comprise culturing, fermentation, harvest filtration, concentration, capture (anion) chromatography, polishing (cation) chromatography, buffer exchange, bioburden reduction and vial fill.

The upstream stage included use of a culture medium in a 1 L bottle containing 400 mL of reduced (in an anaerobic chamber) seed APF culture medium (2% w/v SPTII, 1% w/v yeast extract, (adjusted to pH 7.3 with 1N sodium hydroxide and/or 1N hydrochloric acid prior to autoclaving)) 1% w/v sterile glucose added post autoclaving of culture media). The culture (seed) medium was inoculated with 400 μL of a thawed Clostridium botulinum WCB. Incubation/culturing occurred at 34.5° C.±1.0° C. with 150 rpm agitation in an anaerobic chamber.

When the optical density of the culture medium at 540 nm was 1.8±1.0 AU, the entire contents of the 1 L bottle (approximately 400 mL) were transferred to a 20 L production fermentor containing APF fermentation medium adjusted with 1N sodium hydroxide and/or 1N hydrochloric acid post-steam sterilization to pH 7.3, fermentation medium composed of 3.25% w/v SPTII, 1.2% w/v yeast extract, 1.5% w/v sterile glucose (added post sterilization; sterilization, e.g. at about 122° C. for 0.5 hour). The temperature and agitation were controlled at 35° C.±1° C. and 70 rpm, respectively. Nitrogen overlay was set at 12 slpm and headspace pressure set at 5 psig to maintain an anaerobic environment for cell growth. Fermentation pH and cell density were monitored by pH and online turbidity probes, respectively. The three phases for the production fermentation include exponential growth, stationary, and autolysis phases. Cellular autolysis, which releases active BoNT/A complex into the culture medium, was observed to occur consistently between 35 hours and the end of fermentation. At the end of fermentation, the culture was cooled to 25° C. for harvest.

Once the fermentation medium was cooled to 25° C., the cell debris was separated from the botulinum neurotoxin type A complex containing lysate by depth filtration, first through a 5-0.9 μm nominal retention rating gradient pre-filter to remove cell debris, and then through a positively charged 0.8-0.2 μm nominal retention rating gradient to remove DNA (removal of up to about 80%). Both filters were rinsed together with 20 L of water for injection (WFI) before use. A minimum of 15 L of the filtrate was required for further processing, and any excess material was decontaminated after in-process sampling is complete. The filtrate was stored at 4° C. if not immediately processed by ultrafiltration.

Within a biosafety cabinet (BSC) the filtrate from the harvest step was concentrated from 15 L to 5±0.5 L using a hollow fiber, tangential flow filtration (TFF) membrane from GE Healthcare. The ultrafiltered material was then diluted with 10 mM sodium phosphate pH 6.5 buffer to a final volume of 20 L. This material was purified by use of either two column (anion then cation) or three chromatography columns (anion, cation, and then hydrophobic interaction). The diluted, ultrafiltered harvest material was stored at 4° C. if not immediately processed by purification.

In the Schantz process the culture step is ended and the fermentation step begun based on time and visual observation of culture growth. In contrast, in processes disclosed herein, determination of when to end the culturing step is based on analysis of culture fluid optical density, which ensures that the culture is in the logarithmic growth phase at the time of commencement of the fermentation step, and permits reduction of duration of the culturing step to about 8 hours to about 14 hours. The OD parameter terminated culture step maximized the health of the cultured cells and encouraged robust and abundant botulinum toxin resulting from the fermentation step. The average optical density (at 540 nm) of the culture medium at conclusion of culturing was 1.8 AU. The average duration of the fermentation step 72 hours and the average final turbidity (A890) of the fermentation medium at conclusion of the fermentation step was 0.15 AU. The average amount of botulinum toxin type A complex present (as determined by ELISA) in the 20 L fermentation medium (whole broth) at the end of the fermentation step for was about 64 μg botulinum toxin type A complex/mL fermentation medium.

The harvest step used depth filtration to remove cell debris and nucleic acids, followed by ultrafiltration and dilution to prepare the fermentation medium for the next step in the process. This harvesting/cell debris clearing is fundamentally different from the Schantz harvest process, which uses precipitation by acidification followed by microfiltration and diafiltration to concentrate and exchange buffers in preparation for further processing.

### Example 2

### Production of a Master Cell Bank

Creation of a master cell bank is required for regulatory approval of a biologic. U.S. Pat. No. 8,324,349, incorporated entirely by reference, describes a method of making an animal product free (APF) master cell bank for a BoNT/A producing Clostridium botulinum as follows. A previously established Schantz master cell bank (MCB) was used to create an APF research cell bank (RCB) from which a new APF master cell bank (MCB) and a subsequent working cell bank (WCB) were generated. A research cell bank (RCB) was made from a colony from the Schantz (NAPF) MCB. To remove the animal-derived protein from the MCB vial, the cells were washed twice in APF medium containing 2% w/v SPTII (Soy Peptone type II), 1% w/v yeast extract, and 1% w/v glucose. The cells were plated on APF medium under strict anaerobic conditions using a Modular Atmosphere Controlled System (MACS) anaerobic chamber (anaerobic gases a combination of H₂, CO₂ and N₂.) An isolated colony was further expanded and stored in APF medium containing about 20% glycerol below −135° C.

The APF-MCB was made under GMP conditions by expanding the RCB into oxygen-free APF medium (200 mL, reduced for a minimum of 12 hours in an anaerobic chamber) and cultured in a MACS anaerobic chamber at 34.5° C.±1° C. (stirred at 60 rpm) until the OD₅₄₀ of the culture reached 2.5±1.0 AU. Sterile glycerol was added to the resultant culture to a final concentration of about 20% after which the mixture was transferred into cryovials at 1 mL/vial (APF-MCB vials). The vials were flash frozen in liquid nitrogen, and then stored below −135° C. An APF-WCB was made under GMP conditions by expanding as above. The resultant APF cell banks were characterized for identity, purity, viability and genetic stability.

### Example 3

### BoNT/E Fermentation with Growth Temperature of 20° C.

Clostridium botulinum cells producing BoNT/E are produced using anaerobic conditions similar to those previously described (see, e.g., Frank Gessler, Journal of Biotechnology 119 (2005) 204-211, and Prabhakaran et al., Toxicon 39 (2001) 1515-1531.) In a preferred embodiment, culture medium is animal product free, such as a soy based medium.

The fermentation step in Prabhakaran is described as follows. The culture medium was yeast extract (Difco) 0.5%; Trypticase-peptone (BBL) 2%; Na-thioglycolate 0.025%; and glucose 1% (before autoclaving pH of the medium was adjusted to 6.2). Strain Alaska E-43 stored at −80° C. were inoculums (0.5 mL) for 25 mL of cooked meat media (Difco) incubated at 30° C. (day 1). Next day four 13-l glass carboys (Pyrex) each containing 9500 mL of culture medium (minus glucose) were autoclaved 1 h; each of which then received glucose (100 g in 500 mL water) separately autoclaved for 30 min. The carboys, now each containing 10 L of the complete medium, cooled down overnight to 30° C. (±1° C.) were inoculated (day 3) with ˜2.0 mL of culture actively growing in the cooked meat medium (aliquots of this 48-h-old culture stored at −80° C. up to 7 years have served as starter culture) and incubated undisturbed at 30° C. (±1° C.).

The fermentation step in Gessler is described as follows. The inoculum was grown in 100-mL flasks with 50 mL clostridial medium (CM) which consisted of 0.3% yeast extract, 0.75% meat extract, 0.75% peptone from casein, pancreatically digested, 0.75% peptone from meat, tryptically digested, 0.1% soluble starch, 0.5% d-glucose, 0.5% sodium chloride, 0.3% sodium acetate, 0.05% l-cysteine-HCl. The pH was adjusted to 7.0. The inoculum was incubated overnight at 26° C. in an anaerobic atmosphere (80% N₂, 5% H₂, 15% CO₂). A 10-L fermentor vessel with 1 L CM was inoculated and the pH initially adjusted to 6.8. To ensure an anaerobic atmosphere, the fermentor was supplied with a continuous N₂ overflow. If the bacteria grew well, 9 L of CM were added after ˜24 h to give a final culture volume of 10 L and incubated for another 4 days at 26° C.

In order to slow growth even further, instead of the 30° C. fermentation step temperature used in Prabhakaran or the 26° C. fermentation step temperature used in Gessler, a fermentation step of 20° C.±2° C. is used after the inoculation step. To compensate for the slower growth, the fermentation step would be extended to five (5) days or more.

Purification and activation of BoNT/E would be essentially as previously described (see, e.g., Frank Gessler, Journal of Biotechnology 119 (2005) 204-211 and Prabhakaran et al., Toxicon 39 (2001) 1515-1531.)

### Example 4

### BoNT/E Fermentation with Growth Temperature of 15° C.

Clostridium botulinum cells producing BoNT/E are produced as in Example 3 but with a fermentation step of 15° C.±2° C. is used after the inoculation step. To compensate for the slower growth, the fermentation step is extended to seven (7) days or more.

### Example 5

### BoNT/E Fermentation with Growth Temperature of 10° C.

Clostridium botulinum cells producing BoNT/E are produced as in Example 3 but with a fermentation step of 10° C.±2° C. is used after the inoculation step. To compensate for the slower growth, the fermentation step is extended to seven (7) days.

### Example 6

### BoNT/E Fermentation with Growth Temperature of 5° C.

Clostridium botulinum cells producing BoNT/E are produced as in Example 3 but with a fermentation step of 5° C.±2° C. is used after the inoculation step. To compensate for the slower growth, the fermentation step is extended to seven (7) days

### Example 7

### BoNT/E Fermentation with Inoculation Temperature of 20° C.

Clostridium botulinum cells producing BoNT/E are produced as in any of Examples 4-6 but with an inoculation step of 20° C.±2° C.

### Example 8

### BoNT/E Fermentation with Inoculation Temperature of 20° C.

Clostridium botulinum cells producing BoNT/E are produced as in any of Examples 4-6 but with an inoculation step of 20° C.±2° C.

### Example 9

### BoNT/E Fermentation with Inoculation Temperature of 15° C.

Clostridium botulinum cells producing BoNT/E are produced as in any of Examples 4-6 but with an inoculation step of 15° C.±2° C.

### Example 10

### BoNT/E Fermentation with Inoculation Temperature of 10° C.

Clostridium botulinum cells producing BoNT/E are produced as in any of Examples 4-6 but with an inoculation step of 10° C.±2° C.

### Example 11

### BoNT/E Fermentation with Growth at pH 7.5

Clostridium botulinum cells producing BoNT/E are produced using anaerobic conditions similar to those previously described in an animal product free, soy based medium. In order to prolong growth of the cells, the pH is set to 7.5±0.3 and maintained within this range during growth. Prior to harvesting and lysis of the cells, the pH is adjusted to acidic conditions to avoid denaturing the toxin.

### Example 12

### BoNT/E Fermentation with Growth at pH 8

Clostridium botulinum cells producing BoNT/E are produced using anaerobic conditions similar to those previously described in an animal product free, soy based medium. In order to prolong growth of the cells, the pH is set to 8.0±0.3 and maintained within this range during growth. Prior to harvesting and lysis of the cells, the pH is adjusted to acidic conditions to avoid denaturing the toxin.

### Example 13

### BoNT/E Fermentation with Growth at pH 8.5

Clostridium botulinum cells producing BoNT/E are produced using anaerobic conditions similar to those previously described in an animal product free, soy based medium. In order to prolong growth of the cells, the pH is set to 8.5±0.3 and maintained within this range during growth. Prior to harvesting and lysis of the cells, the pH is adjusted to acidic conditions to avoid denaturing the toxin.

### Example 14

### BoNT/E Fermentation with Growth at pH 9.0

Clostridium botulinum cells producing BoNT/E are produced using anaerobic conditions similar to those previously described in an animal product free, soy based medium. In order to prolong growth of the cells, the pH is set to 9.0±0.3 and maintained within this range during growth. Prior to harvesting and lysis of the cells, the pH is adjusted to acidic conditions to avoid denaturing the toxin.

### Example 15

### Trypsin Minimization in Drug Substance

Clostridium botulinum cells producing BoNT/E were grown under anaerobic conditions similar to those previously described (Prabhakaran et al., (2001) Toxicon 39: 1515-1531.) After the primary recovery and concentration of product, BoNT/E was activated with bovine trypsin as follows: ˜0.2 L of Trypsin Digestion Load was mixed with ˜0.5 L Buffer (0.2M Phosphate, pH 6.0), immobilized TKCK trypsin and filtered at 300 C with a 0.2 μm filtered.

Diafiltration with UF/DF at ambient room temperature over about three hours was used to a concentration factor of three: ˜0.6 L hydrophobic interaction chromatography (“HIC”) Elution Pool (0.05M Phosphate, pH 6.0), ˜1 L Buffer (0.2M Phosphate, pH 6.0), and a hollow fiber filter with a molecular weight cutoff of 50 kDa.

A second diafiltration step at ambient room temperature over about three hours removed additional impurities: ˜0.2 L HIC Elution Pool (0.05M Phosphate, pH 6.0), ˜2 L Buffer (0.05M Tris, pH 8.0), and a hollow fiber filter with a 10 kDa cutoff.

An anion exchange (AEX) column at ambient room temperature over about two hours removed most of the remaining trypsin: ˜0.2 L AEX Load (0.05M Tris, pH 8.0), ˜1 L Buffer A (0.05M Tris, pH 8.0); ˜1 L Buffer B (0.05M Tris, 1M NaCl, pH 8.0), Fractogel TMAE (S) column.

Finally, a third diafiltration step at ambient room temperature over about three hours finished impurity removal: ˜0.1 L AEX NT Elution Pool (0.05M Tris, pH 6.0), ˜1 L Buffer (0.03M Acetate, 0.236M Trehalose, pH 8.0), and a hollow fiber filter with a 10 kDa cutoff.

In additional to bovine trypsin, porcine trypsin could can also be used for activation of BoNT/E.

A fluorescent reporter susceptible to trypsin was added to the BoNT/E DS. Results of incubation over a three-hour time course at 37° C. and compared to a standard curve gave a calculation of 46 to 89 pg/mL trypsin in 90 ng BoNT/E drug substance. Trypsin levels can also be assessed using mass spectroscopy or ELISA.

### Example 16

### GMP Master Cell Bank Using Argon

A glycerol solution was made as a 40% solution in Sterile Water for Irrigation (SWFI), then then further diluted to 20% with soytone/yeast/dextrose (SYD). SYD broth, SYD assay (SYDA) plates and 20% glycerol are to be prepared at least 48 hours prior to use to allow for reduction. A previously established Clostridium botulinum serotype E3, (Alaska E43) clonal primary seed bank (PSB) was used to create a master cell bank (MCB). 10 μl inoculating loops were used to plate cell suspension from the PSB vial on reduced SYDA plates and incubated for 30±2° C. for 5 to 7 days to confirm purity. To minimize animal product, 1 mL of the PSB was aseptically transferred to a reduced SYD growth tube with an inoculum ratio of 1:10 and placed in an anaerobic jar and incubated at 30±2° C. for about 30 hours. 2 mL of the growth tube was aseptically transferred to a MCB growth flask and placed in an anaerobe jar and incubate at 30±2° C. for 24±4 hours. The MCB culture of SYD and 20% glycerol:SYD was gently mixed with an overlay of argon for 30-45 minutes. 1.2 mL of MCB was then transferred, aseptically, into cryovials while maintaining an overlay of sterile, filtered argon. The cryovials were placed in cryogenic storage boxes and lyophilized at −40° C., then transferred to storage at ≤−65° C.

### Example 17

### Fermentation Using Argon

In a prophetic embodiment, BoNT/E is produced using conditions similar Frank Gessler, Journal of Biotechnology 119 (2005) 204-211, or Prabhakaran et al., Toxicon 39 (2001) 1515-1531, but using argon instead of N₂, H₂, and/or CO₂.

### Example 18

### Use of Botulinum Toxin Type E to Treat Axillary Hyperhidrosis

A 44-year old male complains of excessive axillary perspiration. His doctor recommends a Minor's starch iodine test. In the Minor's test, the skin is cleaned, shaved, and dried. A 3.5% iodine in alcohol solution is applied to the skin of the affected area, and starch flour is sprinkled on top. The color changes to a dark violet as sweat contacts the iodine-starch mixture. This indicates a positive sweat test and yields a diagram of the distribution of active eccrine glands.

His doctor diagnoses axillary hyperhidrosis, and prescribes injections of BoNT/E to in the pattern indicated by the Minor's test to provide rapid relief. The injections are made subcutaneously (s/c) to the underarm in a grid-like pattern approximately every 1-2 cm apart. Each injection contains 10 units of type E neurotoxin.

The patient is scheduled to return to the doctor after a week for results evaluation as well as supplemental administrations if necessary.

### Example 19

### Use of Botulinum Toxin Type E to Treat Glabellar Lines (GL)

This first-in-human, randomized, double-blinded, placebo-controlled, ascending dose cohort study enrolled 42 subjects who received EB-001 (a BoNT/E composition disclosed herein) (N=35) or placebo (N=7). The efficacy primary outcome was the proportion of subjects with a 2-grade investigator-rated (IR-2) improvement in GL severity at maximum frown. Safety evaluations included adverse events (AEs), laboratory tests, and physical examinations. An IR-2 response was observed starting in the third cohort (EB-001), with increased rates observed at higher doses. Onset of clinical effect was within 24 hours, with a duration ranging between 14 and 30 days for the highest doses. AE incidence was low, with the most common being mild to moderate headache. There were no serious AEs or ptosis, and no clinically significant changes in other safety assessments.

In this clinical study in GL, EB-001 showed favorable safety and tolerability, and dose dependent efficacy with an 80% response rate at the highest dose. EB-001 maximum clinical effect was seen within 24 hours and lasted between 14 and 30 days. This differentiated EB-001 profile supports its development for aesthetic and therapeutic applications where fast onset and short duration of effect are desirable.

Botulinum neurotoxins, which inhibit the pre-synaptic release of acetylcholine, are among the most potent molecules in nature. When injected into muscles, Botulinum neurotoxins inhibit neuromuscular transmission and produce dose-dependent local muscle relaxation. Purified Botulinum neurotoxins, including serotypes A and B have been developed as injectable drugs and are widely used to treat a variety of neuromuscular conditions. Botulinum neurotoxin serotype E is a novel serotype that has not been developed for clinical use to date. Botulinum toxin type E has the fastest onset and the shortest duration of action of all the Botulinum neurotoxins. Type E has similar domain structure to type A, consisting of 2 protein chains, a 100 kDa heavy chain and a 50 kDa light chain linked by a disulfide bond.2 Type E inhibits neuromuscular transmission by cleaving the same presynaptic vesicular protein (synaptosomal associated protein 25) as type A, but at a different cleavage site. Two binding sites on motor axons mediate the high affinity recognition of nerve cells by Botulinum neurotoxins. Binding is mediated first by cell surface gangliosides and then by specific protein receptors. These receptors are found on motor axon terminals at the neuromuscular junction. Botulinum toxin types A and E have both been shown to bind the specific receptor synaptic vesicle protein 2, and only these two serotypes share this receptor. This was the first clinical study to evaluate the safety and efficacy of ascending doses of Botulinum toxin type E in subjects with GL.

This study was a first-in-human evaluation of the safety and efficacy of EB-001 and focused on the treatment of moderate to severe GL. EB-001 is a proprietary purified form of Botulinum toxin type E, formulated as a liquid for injection (Bonti, Inc., Newport Beach, Calif., USA). This was a randomized, double-blinded, placebo-controlled, ascending-dose cohort study conducted at 2 expert clinical centers (Steve Yoelin, MD Medical Associates, Newport Beach, Calif., USA; Center for Dermatology Clinical Research, Fremont, Calif., USA). This study was approved by an Institutional Review Board (Aspire Institutional Review Board, Santee, Calif., USA) and was conducted in accordance with the guidelines set by the Declaration of Helsinki. Written informed consent was received from all subjects prior to their participation.

A total of 42 healthy toxin-naïve male and female subjects, ages 18 to 60 years, were enrolled in the study. Each subject's participation was to last approximately 6 weeks. The main inclusion criteria were: the presence of bilaterally symmetrical GL of moderate to severe rating at maximum frown, sufficient visual acuity without the use of eyeglasses (contact lens use acceptable) to accurately assess their facial wrinkles, and the ability to conform with study requirements. The main criteria for exclusion were: any uncontrolled systemic disease or other medical condition, any medical condition that may have put the subject at increased risk with exposure to Botulinum neurotoxin (including diagnosed myasthenia gravis, Eaton-Lambert syndrome, amyotrophic lateral sclerosis, or any other condition that interfered with neuromuscular function), current or prior Botulinum neurotoxin treatment, known immunization or hypersensitivity to Botulinum neurotoxin, pre-specified dermatological procedures within 3 to 12 months of the study (non-ablative resurfacing, facial cosmetic procedures, topical/oral retinoid therapy, etc.), and prior periorbital surgery or treatment. Women were not enrolled if they were pregnant, lactating, or planning to become pregnant. Men with female partner(s) of childbearing potential were enrolled only if they agreed to use dual methods of contraception for 3 months following dosing.

At Screening, subject demographics, medical history, and prior and concomitant medications were recorded and an alcohol/drug screen was performed. Standardized facial photography was performed at Baseline prior to treatment, and at every follow-up visit through the end of the study, but the photographs were not used for efficacy evaluations.

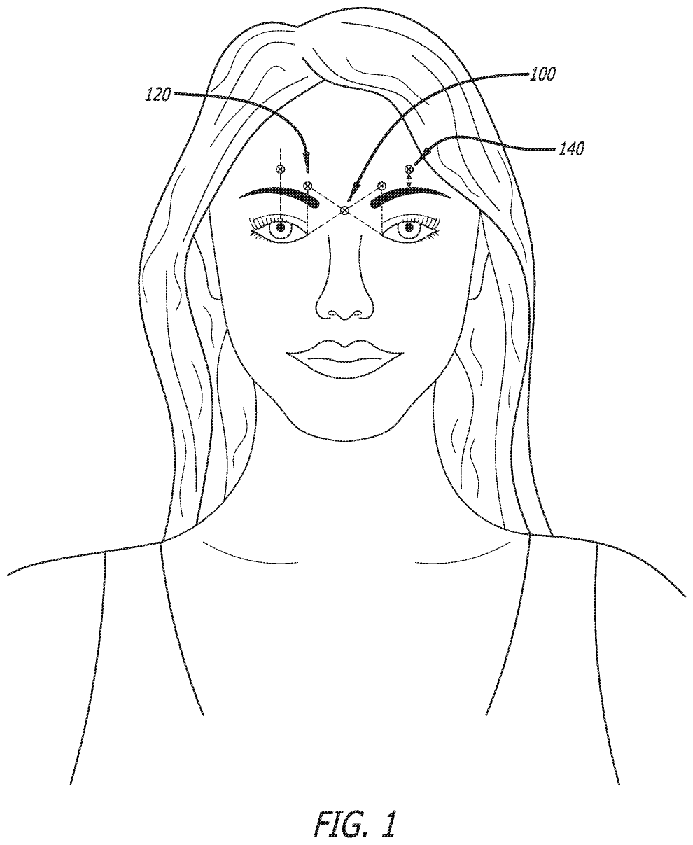

Seven cohorts (6 subjects per cohort) were enrolled and received ascending doses of EB-001 or placebo in a 5:1 ratio. The maximum recommended starting dose (with a 10-fold safety factor) in this first-in-human study was developed based on the no observed adverse effect levels from a preclinical safety and toxicity study (unpublished data). From this, a base dose (Cohort 1) was calculated and determined to be sub-efficacious, and Cohorts 2 to 7 received 3, 9, 12, 16, 21, and 28 times the base dose, respectively. This represented sub-efficacious to maximum-efficacious doses of EB-001. The total dose was delivered at 5 injection sites in equal volumes (0.1 mL per site into the procerus, left and right medial corrugators, and left and right lateral corrugators) in a standardized fashion (see FIG. 1). The spacing of injections into the lateral corrugators was approximately 1 cm above the supraorbital ridge. EB-001 was supplied in a sterile solution for injection in a 5-mL vial. The placebo was supplied in identical vials without EB-001.

Each subject completed visits at Screening (Day −30 to −1), Baseline/Injection (Day 0), Days 1, 2, 7, 14, and 30 (end of study), and Day 42 (final safety follow-up).

Safety was evaluated by adverse events (AEs), laboratory testing, electrocardiograms (ECGs), physical examinations, vital signs (pulse rate, respiratory rate, and blood pressure), urine pregnancy tests (for women of childbearing potential), and focused neurologic examinations to evaluate for the potential spread of Botulinum neurotoxin. Treatment-emergent AEs (TEAEs) were defined as any AE that started or worsened in severity after exposure to study treatment. AEs and TEAEs were summarized by system organ class and preferred term using the Medical Dictionary for Regulatory Activities (MedDRA, version 19.0). Serious AEs (SAEs, or AEs that fulfilled regulatory criteria for medical seriousness), and discontinuation due to AEs were also evaluated. Severity of AEs was recorded as mild, moderate, severe, or life threatening. Before enrollment of each dosing cohort, a safety data review committee met to analyze all safety data from the previous cohort(s).

At Screening, Baseline, and Days 1, 2, 7, 14, and 30, the subject's GL were assessed at maximum frown and at rest using the Facial Wrinkle Scale (FWS). Evaluations were completed by the investigator and the subject. The FWS is a widely accepted measure used for the evaluation of facial line severity. In the present study, the 4-point scale indicating severity of GL was as follows: 0=none, 1=mild, 2=moderate, 3=severe. Subjects were considered as treatment responders if they achieved at least a 2-grade improvement (reduction) based on the investigator's FWS assessment (IR-2). The primary efficacy variable was the proportion of IR-2 responders at maximum frown at any post baseline visit through Day 30. An additional efficacy endpoint of interest was the proportion of responders achieving an investigator-assessed FWS grade of none or mild at Days 1, 2, 7, 14, or 30 (analyzed by visit).

Two analysis populations were pre-specified, a safety and an efficacy population. Subjects receiving placebo were pooled for all analyses. The safety population included all subjects who received study treatment and had at least 1 safety assessment thereafter. All TEAEs and SAEs were summarized by treatment group. All safety parameters, including laboratory testing, ECGs, physical exams, vital signs, urine pregnancy tests, and focused neurologic examinations, were reviewed and evaluated for clinical significance by the investigators. The efficacy population was the modified intent-to-treat (mITT) population, defined as all randomized subjects who received at least 1 dose of study treatment and had at least 1 post baseline efficacy assessment. Analyses of demographics and baseline characteristics were performed on the mITT population. Medical history was based on the safety population and coded using MedDRA and summarized by system organ class and preferred term. Prior and concomitant medications were based on the safety population and coded using the World Health Organization Anatomical Therapeutic Chemical classification index and summarized by drug class and treatment group. Efficacy analyses were performed using the mITT population. FWS grades were summarized by treatment and study day using frequency counts and rates of response (%). An analysis comparing the proportion of IR-2 responders in each EB-001 cohort versus placebo (pooled) was performed using Fisher's exact test with a 0.05 level of significance.

Of the 59 subjects who were screened for the study, 43 were enrolled into 1 of 7 cohorts. One subject did not receive treatment, and consequently 42 subjects were included in the mITT and safety populations (35 treated with EB-001 and 7 treated with placebo). Forty-one subjects completed the study, with 1 subject lost to follow-up. The demographic and baseline characteristics of the mITT population are displayed in Table 1. The mean (range) ages of subjects for the EB-001 (pooled) versus placebo (pooled) groups were 47.9 (22 to 60) and 50.4 (32 to 57) years, respectively. The majority of subjects were female (EB-001=91.4%; placebo=85.7%) and white (71.4% for both groups). The baseline mean (standard deviation [SD]) investigator-assessed GL at maximum frown were 2.6 (0.50) and 2.9 (0.38) for the EB-001 and placebo groups, respectively. The EB-001 and placebo groups were well balanced with no substantial between-group differences.

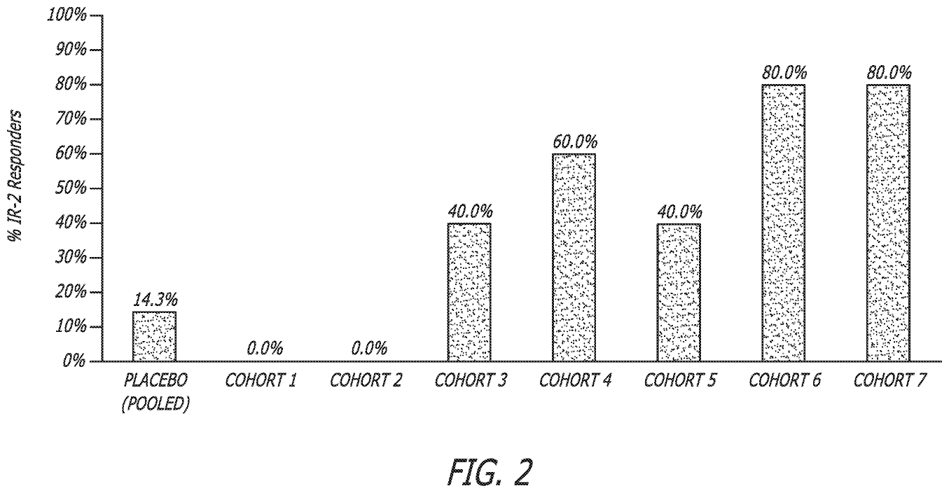

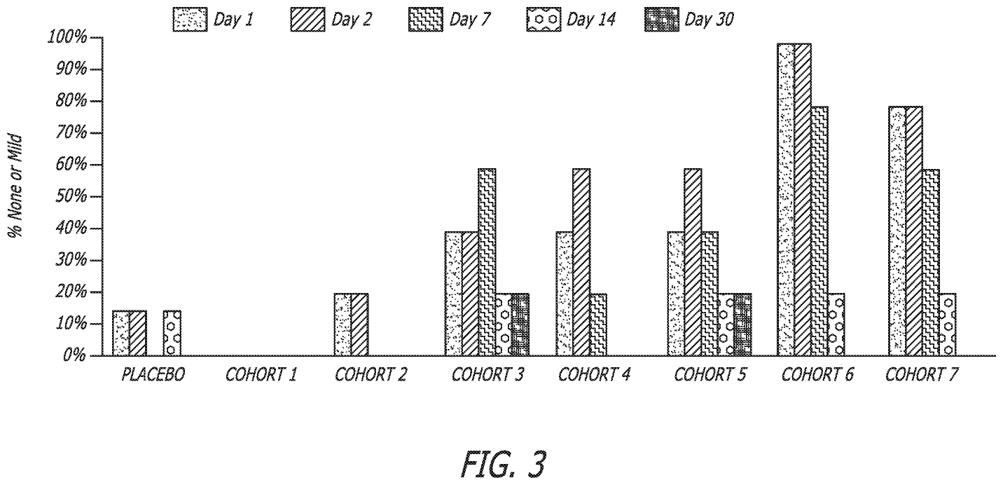

The proportions of subjects in the mITT population achieving an IR-2 response for GL severity at maximum frown at any postbaseline visit through Day 30 are presented by dose cohort in FIG. 2. In Cohort 3, 40% of subjects were IR-2 responders. This responder rate was the same or greater in all higher dose cohorts, with Cohorts 6 and 7 having 80% IR-2 responders. Cohorts 6 and 7 demonstrated significantly greater percentages of IR-2 responders versus placebo (P=0.046). FIG. 3 summarizes the proportions of subjects in each cohort with investigator-assessed FWS grades of none or mild GL at maximum frown, at any post baseline visit through Day 30. Cohorts 2 to 7 (inclusive) had greater percentages of responders versus placebo, with rates of 60% to 100% achieved for Cohorts 3 and higher. In Cohorts 3 to 7, most none or mild responses were observed at Days 1, 2, and/or 7. One responder (20%) was observed at Day 14 in Cohorts 3, 5, 6 and 7 and at Day 30 in Cohorts 3 and 5. The safety results support the safety of all evaluated doses of EB-001, administered as IM injections, in this population. No clinically significant changes from baseline in neurologic examinations, ECGs, physical examinations, or laboratory tests were observed for any subject.

Five subjects treated with EB-001 reported TEAEs, and none in placebo group. No SAEs were reported and no TEAE led to discontinuation of the study. All TEAEs were mild or moderate in severity. The events of sore throat and flu like symptoms were considered unrelated to treatment. Three subjects reported TEAEs of headache, 1 of which was considered related to treatment. There was no dose-related increase in the incidence of headaches. There were no events of ptosis or other TEAE possibly related to spread of toxin.

To our knowledge, this is the first controlled clinical trial of a Botulinum toxin type E product in any aesthetic or therapeutic use. This first-in-human study of EB-001, a novel purified form of Botulinum toxin type E administered IM, fulfilled its objectives of evaluating the safety, tolerability, and efficacious dose-range of EB-001. A dose response was observed, with greater proportions of treatment responders in the higher dosing cohorts of EB-001. An IR-2 response was observed starting with Cohort 3 and increased in higher dose cohorts, suggesting that the efficacious dose range of EB-001 may be at doses used in Cohorts 4 to 7. Cohorts 6 and 7 had 80% IR-2 responders, a response rate similar to approved Botulinum toxin type A products. Subjects achieving none or mild FWS grades were observed starting at Cohort 2. In terms of onset of effect, treatment response was observed as early as 24 hours following dosing, which supports prior reports suggesting that Botulinum toxin type E has a faster onset than type A.

Regarding the duration of effect defined as the proportion of responders with a none or mild rating, an effect was observed through Day 14 in 1 subject in most of the 5 higher dose cohorts, and through Day 30 in 1 subject in 2 of the 5 higher dose cohorts. All doses of EB-001 showed good tolerability with no local injection site reactions. There were no SAEs or severe TEAEs reported, and no discontinuations due to a TEAE. The most common TEAE of headache was mild or moderate in severity, and there were no other treatment related AEs. There were no events of ptosis at any dose levels, and no events potentially related to spread of toxin. Therefore, the clinical safety and tolerability profile seems favorable in this study. The efficacy and safety profiles of EB-001 are promising and support the potential of EB-001 as a unique treatment option in the treatment of GL and other facial aesthetic uses. The fast onset can fulfill an unmet need for individuals seeking a rapid treatment for facial wrinkles before unexpected social or professional events. The limited duration of effect can be beneficial for individuals who may be considering first time use of a Botulinum neurotoxin treatment, and are unwilling to make a longer-term commitment. An EB-001 treatment would allow them to assess the aesthetic effect over a shorter duration of effect compared with the 12-week duration of effect of Botulinum toxin type A products. In this first clinical study in subjects with GL, EB-001 showed favorable safety and tolerability in all cohorts. Five out of the 7 cohorts showed numerically higher response rates compared to placebo, supporting the efficacy of EB-001 in the reduction of GL severity. The 2 highest doses provided an 80% response rate, similar to approved Botulinum toxin type A products. In contrast to the known time course of type A products, the clinical effect of EB-001 was seen within 24 hours (onset) and lasted between 14-30 days (duration). This differentiated clinical profile supports the future development of EB-001 for facial aesthetic and key therapeutic uses, where fast onset and short duration of effect are desirable.

TABLE S-1
Dose Escalation Scheme

| | Total | | Doses at | Dose at |
|---|---|---|---|---|
| | EB-001 | Dose at | Medial | Lateral |
| | Dose | Procerus | Corrugators | Corrugators |
| Cohort¹ | (ng)² | (ng) | (ng) | (ng) |
| 1 | 0.1 | EB-001 | EB-001 into | EB-001 into |
| | | (0.02) | right and left | right and left |
| | | | corrugators | corrugators |
| | | | (0.02 each) | (0.02 each) |
| 2 | 0.3 | EB-001 | EB-001 into | EB-001 into |
| | | (0.06) | right and left | right and left |
| | | | corrugators | corrugators |
| | | | (0.06 each) | (0.06 each) |
| 3 | 0.9 | EB-001 | EB-001 into | EB-001 into |
| | | (0.18) | right and left | right and left |
| | | | corrugators | corrugators |
| | | | (0.18 each) | (0.18 each) |
| 4 | 1.2 | EB-001 | EB-001 into | EB-001 into |
| | | (0.24) | right and left | right and left |
| | | | corrugators | corrugators |
| | | | (0.24 each) | (0.24 each) |
| 5 | 1.6 | EB-001 | EB-001 into | EB-001 into |
| | | (0.32) | right and left | right and left |
| | | | corrugators | corrugators |
| | | | (0.32 each) | (0.32 each) |
| 6 | 2.1 | EB-001 | EB-001 into | EB-001 into |
| | | (0.42) | right and left | right and left |
| | | | corrugators | corrugators |
| | | | (0.42 each) | (0.42 each) |
| 7 | 2.8 | EB-001 | EB-001 into | EB-001 into |
| | | (0.56) | right and left | right and left |
| | | | corrugators | corrugators |
| | | | (0.56 each) | (0.56 each) |

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present disclosure, which is defined solely by the claims. Accordingly, embodiments of the present disclosure are not limited to those precisely as shown and described.

Certain embodiments are described herein, comprising the best mode known to the inventor for carrying out the methods and devices described herein. Of course, variations on these described embodiments will become apparent to those of ordinary skill in the art upon reading the foregoing description. Accordingly, this disclosure comprises all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

Groupings of alternative embodiments, elements, or steps of the present disclosure are not to be construed as limitations. Each group member may be referred to and claimed individually or in any combination with other group members disclosed herein. It is anticipated that one or more members of a group may be comprised in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope otherwise claimed. No language in the present specification should be construed as indicating any non-claimed element essential to the practice of embodiments disclosed herein.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present disclosure so claimed are inherently or expressly described and enabled herein.

1. A method of manufacturing botulinum neurotoxin serotype E comprising a fermentation step, wherein the temperature of the fermentation step is between 5° C. and 20° C.

2. A method of manufacturing botulinum neurotoxin serotype E comprising a fermentation step, wherein the temperature of the fermentation step is less than 20° C.

3. The method of claim 2, wherein the temperature of the fermentation step is 15° C., ±2° C.

4. The method of claim 3, wherein the duration of the fermentation step is seven (7) days.

5. The method of claim 2, wherein the temperature of the fermentation step is 10° C., ±2° C.

6. A method of manufacturing botulinum neurotoxin serotype E comprising a fermentation step, wherein the temperature of the fermentation step is between 10° C. and 15° C.

7. A method of manufacturing botulinum neurotoxin serotype E comprising a fermentation step, wherein the pH of the growth step is between 7.2 and 9.3.

8. The method of claim 7, wherein the pH is 7.5±0.3.

9. The method of claim 7, wherein the pH is 8.0±0.3.

10. The method of claim 7, wherein the pH is 8.5±0.3.

11. The method of claim 7, wherein the pH is 9.0±0.3.

12. A method of manufacturing botulinum neurotoxin serotype E comprising a fermentation step, wherein the pH of the growth step is 7.5 or above.

13. The method of claim 12, wherein the pH is 8.0 or above.

14. The method of claim 12, wherein the pH is 8.5 or above.

15. A composition comprising a drug substance botulinum neurotoxin serotype E and a trypsin at a level between 40 pg and 90 pg in 90 ng neurotoxin.

16. A method of creating a Clostridium botulinum master cell bank comprising the use of argon to maintain anaerobic conditions.

17. The method of claim 16, wherein the Clostridium botulinum produces botulinum neurotoxin serotype E.

18. The method of claim 16, wherein the Clostridium botulinum produces botulinum neurotoxin serotype A.

19. A method of manufacturing botulinum neurotoxin comprising fermentation of Clostridium botulinum, wherein the anaerobic conditions are maintained using argon.

20. The method of claim 19, wherein the botulinum neurotoxin is botulinum neurotoxin serotype E.

21. The method of claim 19, wherein the botulinum neurotoxin is botulinum neurotoxin serotype A.
